(19) United States
(12) Patent Application Publication
(10) Pub. No.: US 2013/0216592 A1
(43) Pub. Date: Aug. 22, 2013

(54) PARTICLES CONSISTING OF A CHITOSAN POLYELECTROLYTE COMPLEX AND OF AN ANIONIC POLYSACCHARIDE, AND HAVING IMPROVED STABILITY

(71) Applicants: Thierry Delair, Echalas (FR); Bernard Verrier, Mornant (FR); Franck Gaudin, Lyon (FR)

(72) Inventors: Thierry Delair, Echalas (FR); Bernard Verrier, Mornant (FR); Franck Gaudin, Lyon (FR)

(73) Assignees: Universite Claude Bernard Lyon 1, Villeurbanne Cedex (FR); Universite Jean Monnet Saint- Etienne, Saint Etienne Cedex 2 (FR); Institut National Des Sciences Appliquees De Lyon, Villeurbanne Cedex (FR); Centre National De La Recherche Scientifique, Paris Cedex 16 (FR)

(21) Appl. No.: 13/812,179

(22) PCT Filed: Jul. 26, 2011
(86) PCT No.: PCT/FR2011/051794

§ 371 Date: Mar. 14, 2013

(30) Foreign Application Priority Data
Jul. 30, 2010 (FR) 1056300

(51) Int. Cl. A61K 9/14; A61K 45/06; A61K 47/36

(52) U.S. Cl. CPC A61K 9/146; A61K 47/36; A61K 45/06
USPC 424/400; 514/777

(57) ABSTRACT

The present invention relates to positively charged particles consisting of a chitosan polyelectrolyte complex and of an anionic polymer, characterized in that the chitosan has a degree of acetylation (DA) in the range of 35 to 49% and a mean molar mass by weight (Mw) in the range of 55 to 150 kg/mol, as well as to a method for preparing same.

The present invention relates to the general technical field of particles composed of biodegradable polymers. More precisely, the present invention relates to positively charged particles consisting of a polyelectrolyte complex of chitosan and an anionic polymer, as well as to a method for preparing such particles.

Today, particles, in the general sense of the term, are used in a large number of applications in the fields of chemistry, cosmetics, food processing and life sciences, among others. For biological applications and/or cosmetics, and, in particular, with the aim of minimizing the impact of the use of such nanoparticles, much work in recent years has been devoted to the elaboration of particles from raw materials from biomass (polysaccharides, proteins) and, in particular, biodegradable polymers.

In the context of the prior work, some of the inventors of the present patent application were interested in the manufacture of particles by the formation of polyelectrolyte complexes of polymers from biomass. In particular, the publication by Schatz et al. in Langmuir 2004, 20(18), 7766-7778 demonstrated that it was possible to form micron- and submicron-scale particles by the addition of an aqueous solution of a polycation (or polyanion) to an aqueous solution of a polyanion (or polycation), under simple stirring, the order of addition not being a limiting factor. In 2008 (Drogoz et al. Biomacromolecules, 2008, 9(2), 583-591), they further showed that such particles could be associated with a protein and had an adjuvant capacity in a vaccine application.

Nevertheless, their more recent work (Weber et al. Journal of Biomedical Materials Research Part A, 2010, 93A(4), 1322-1334) on the preparation of particles by the formation of polyelectrolyte complexes between chitosan (polycation) and dextran sulfate (polyanion) showed that such particles did not have satisfactory stability in a medium rich in salts and/or a pH corresponding to physiological pH. It should be recalled that chitosan is a partially or completely deacetylated chitin derivative. Chitosan is thus a copolymer of N-acetylglucosamine and glucosamine joined by a β-1,4 glycosidic bond. Its various forms are notably characterized by their degree of acetylation (DA) and their average molar mass by weight (Mw). Dextran sulfate is a polymer of repeating glucose units in which certain hydroxyl functional groups are sulfated. These two polymers are represented below.

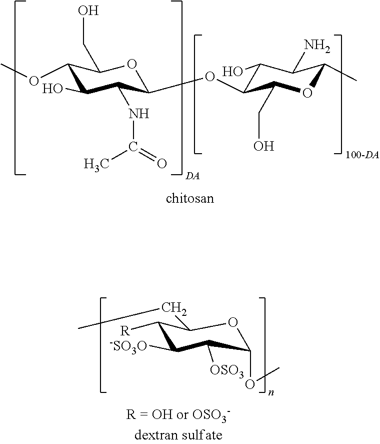

In an aqueous medium, in particular a slightly acidic medium, chitosan is found in a polycationic form, by protonation of these NH₂ functional groups, and thus chitosan is described as a polycation. Furthermore, chitosan is available in the form of various salts, in particular in its hydrochloride form.

The conclusions of this 2010 publication link the stability of the particles to the degree of acetylation of the chitosan and emphasize that chitosan with a DA≧50% leads to predominant hydrophobic interactions and promotes the association of polymer chains. Among the tests conducted, only one carried out with a chitosan of DA equal to 51% and of Mw equal to 150,000 g/mol and a dextran sulfate of Mw equal to 10,000 g/mol led to particles having stability for at least 6 days. But, when the dextran sulfate used has a Mw of 500,000 g/mol, stability is less than 24 hours.

Thus, it is clear that such a problem of stability is a limiting factor for a large number of applications, and in particular for biological applications (therapeutics, diagnostics, cosmetics, etc.). This flocculation leads to variations in the properties of the colloid and thus changes the capacities of transport, encapsulation and adsorption of active ingredients and modifies the interactions of these particles with their environment (e.g., cells, organs, tissues).

The publication by PING-H GGARD M et al. in Gene Therapy, MacMillan Press Ltd, Basingstoke, GB, vol. 8 Jan. 2001, pages 1108-1121 describes a method for preparing positively charged particles of chitosan and plasmid DNA which are prepared only in pure water. Furthermore, this document provides no comment on the colloidal stability in a physiological medium of the particles obtained.

The document EP 1 774 971 describes nanoparticles comprising chitosan, heparin and optionally a polyoxyethylenated derivative, in which the nanoparticles are obtained by virtue of a crosslinking agent enabling the ionic crosslinking of heparin and chitosan. In this document, no information is provided as to the degree of acetylation of the chitosan.

The document WO 2008/003329 describes nanoparticles comprised of chitosan and small interfering RNA (siRNA). siRNA are compounds of low molar mass that constitute a very specific case and no mention is made in this document of stability in physiological medium.

Other teams also noted the lack of stability of particles containing chitosan or chitosan derivatives:
patent application WO 2008/093195, which describes particles comprising a ribonucleic acid, chitosan and a polyanion and which have a negative zeta potential, reports that chitosan-based particles with a positive surface charge are unstable in the presence of salts and protein (page 3, lines 20-25) and thus proposes to create anionic particles,
patent application WO 2006/064331 indicates that chitosan-based polyelectrolyte complexes are unstable in the presence of salts (page 2, lines 10-20) and at physiological pH due to the instability of chitosan at physiological pH (page 3, lines 2-4) and proposes polyelectrolyte complexes that associate a polyanion not with chitosan but with a quaternary chitosan derivative such as N-trimethyl chitosan, N-triethyl chitosan or N-tripropyl chitosan,
U.S. Pat. No. 7,381,716 proposes cationic particles containing chitosan and poly-glutamic acid, whose stability is shown only in deionized water. Moreover, FIG. 7a of this document, which represents images by fluorescence microscopy of the particles obtained, shows the formation of aggregates, in cell culture medium. It should also be noted that in the examples it is specified that the chitosan used is of low Mw and of DA=15%.

It would also be proper to cite patent application US 2008/0254078, which describes nano- and micro-particles consisting of a binary system using chitosan and polyanionic polysaccharides carrying carboxymethyl groups, sulfate groups or carboxy plus sulfate groups. No data is specified concerning the DA of the chitosan used and the only specific data in the examples regarding the Mw of the chitosan used is very slight and is 6,000 g/mol. Although one of the aims of this patent application is to provide stable particles, the stability over time of the particles obtained is not demonstrated.

In this context, there is thus a need for chitosan-based particles having improved stability and for a method for preparing such particles.

Also, the present invention proposes positively charged particles consisting of a chitosan polyelectrolyte complex and of an anionic polymer in which the chitosan has a degree of acetylation (DA) in the range of 35 to 49% and a mean molar mass by weight (Mw) in the range of 55 to 150 kg/mol. Notably, the chitosan has a degree of acetylation (DA) in the range of 45 to 48% and a mean molar mass by weight (Mw) in the range of 70 to 130 kg/mol. Such particles have satisfactory stability under conditions of physiological pH or of a concentration in monovalent salt, such as NaCl.

According to another of its aspects, the invention relates to positively charged particles consisting of a chitosan polyelectrolyte complex and of an anionic polymer, characterized in that the chitosan is selected so that the particles remain stable at room temperature without particular stirring in aqueous media containing physiological concentrations of salt (thus at least equal to 150 mM of monovalent salt) or having physiological pH (i.e., near 7.4), with a solids content (mass of polymers adjusted to 100 ml of dispersion) between 0.01% and 5%, preferentially between 0.1% and 2%. In particular, the stability of the particles is recorded at room temperature, when they are redispersed, after centrifugation, with a solids content (total mass of polymers adjusted to 100 ml of dispersion) between 0.01% and 5%, preferentially between 0.1% and 2%, in water containing 150 mM NaCl or in PBS buffer (pH 7.4), for a period at least equal to 20 days, preferably at least equal to 45 days. The expression “room temperature” refers to a temperature in range of 18-25° C., and in particular equal to 22° C. The particles are considered stable when the variation in their average diameter in relation to D₀ (average diameter of the particles dispersed in the medium, just after elaboration) is less than or equal to 40%, preferentially less than 30%, and even more preferentially less than 20%.

The present invention also has as an aim a method for preparing the above-defined particles which includes the following steps:

a) have available an aqueous solution of chitosan, and, in particular, of a chitosan with a degree of acetylation (DA) in the range of 35 to 49% and a mean molar mass by weight (Mw) in the range of 55 to 150 kg/mol,

b) have available an aqueous solution of anionic polymer,

c) add one of these solutions to the other solution so as to obtain a colloidal solution of positively charged particles consisting of a chitosan polyelectrolyte complex and of the anionic polymer.

The following description will make it possible to better understand the invention.

In the context of the invention, the inventors have demonstrated that it is possible to obtain chitosan-based particles with very high stability and, in particular, stability for a period at least equal to 20 days, preferably at least equal to 45 days, when the particles are dispersed with a solids content of 0.01% to 5%, and preferentially of 0.1% to 2% (total mass of polymers adjusted to 100 ml of dispersion) in water containing 150 mM NaCl or in phosphate buffered saline (PBS; such as, for example, Invitrogen™/GIBCO® PBS, pH 7.4, 1×, lot 712299), by forming a polyelectrolyte complex whose overall charge is positive, by association of a polyanion and a chitosan with a particular DA and Mw. The choice of a chitosan with a degree of acetylation (DA) in the range of 35 to 49%, in particular in the range of 44 to 48%, and a mean molar mass by weight (Mw) in the range of 55 to 150 kg/mol, preferably in the range of 70 to 130 kg/mol, makes it possible to achieve such stabilities. The following examples detail the techniques for measuring DA and Mw, taken as reference, in the context of the invention.

Any type of anionic polymer with sulfate functional groups, carboxymethyl functional groups or carboxylic acid and sulfate functional groups, for example, can be used. Such polymers will in particular belong to the polysaccharide family. For example, the anionic polymer can be selected from hyaluronic acid, dextran sulfate, cellulose sulfate, chondroitin sulfate, heparan sulfate, dermatan sulfate, keratan sulfate, alginates, pectins, carboxymethyl dextran, carboxymethyl amylose, carboxymethyl cellulose, carboxymethyl beta-cyclodextrin, heparin, polystyrene sulfonate, linear or branched water-soluble synthetic homo- or co-polymers containing at least one anionic monomer having either a carboxyl functional group (e.g., acrylic acid, methacrylic acid and salts thereof) or a sulfonic acid functional group (e.g., 2-acrylamido-2-methylpropanesulfonic acid (AMPS) and salts thereof) and optionally one or more nonionic monomers well-known to the person skilled in the art. Nevertheless, dextran sulfate is preferred. Ideally, the average molar mass of the polyanions is not a factor limiting stability. An anionic polymer, and notably dextran sulfate, with an average molar mass by weight in the range of 5 to 5,000 kg/mol, preferably in the range of 5 to 1,000 kg/mol, for example, is used.

The particles according to the invention are positively charged. In particular, the ratio between the number of charges of chitosan and the number of charges of the anionic polymer (n⁺/n⁻) is in the range of 1.05 to 5, preferably in the range of 1.5 to 3.

The particles of the invention are essentially spherical. They can be micron-, submicron- or nanometer-scale particles. Notably, the particles have an average diameter in the range of 50 nm to 50 μm, preferably in the range of 150 nm to 5 μm. The average diameter of the particles can be measured according to various methods known to the person skilled in the art. In the context of the invention, it corresponds to the average hydrodynamic diameter obtained by the quasi-elastic light scattering method and data processing using the cumulants method. A set of particles is also characterized by a polydispersity index corresponding to the formula μ2/[Γ]² where μ2 is the second cumulant of the correlation function resulting from the analysis of the data by the cumulants method and [δ]² is the average rate of decline. A polydispersity index of less than or equal to 0.05 is characteristic of a population of similar size (monodisperse) whereas an index between 0.05 and 0.15 is representative of a wider range of sizes (polydisperse) (Coombes, A. G. A.; Scholes, P. D.; Davies, M. C.; Ilium, L.; Davis S. S. Biomaterials 1994, 15, 673). Advantageously, the particles of the invention have a polydispersity index in the range of 0.01 to 0.25, preferably in the range of 0.05 to 0.2. Such a polydispersity index can be obtained directly by the method of the invention, without the implementation of a filtration step or another fractionation process.

The particles of the invention can be obtained by the addition of an aqueous solution of the chitosan or the anionic polymer to an aqueous solution of the other polymer (chitosan or anionic polymer), said solutions being, for example, at a pH in the range of 2 to 9, preferentially in the range of 3 to 8. Advantageously, at least one of said solutions (or both) contains a monovalent salt at most at a concentration of 400 mM, preferentially at most at a concentration of 150 mM, for example in the form of NaCl. The presence of one such salt makes it possible to stabilize the ionic strength of the medium during the particle elaboration process. To carry out the formation of the particles of the invention, the chitosan and the anionic polymer are solubilized separately, by stirring, in a solution containing a monovalent salt at a concentration between, for example, 0.05 and 150 mM, preferentially between 10 and 70 mM, and even more preferentially between 30 and 60 mM. The chitosan is placed in solution by protonation of its amine functional groups by means of a solution containing, among other things, a strong or weak acid (notably hydrochloric acid or acetic acid). It is also possible to use chitosan acetate or hydrochloride. Each polyelectrolyte is dissolved at a mass concentration (w/v) between, for example, 0.01% and 0.5%, preferentially between 0.05% and 0.3%, and even more preferentially between 0.05% and 0.2%. After dissolution, the pH of each solution is, preferably, adjusted to a value between 2 and 8, preferentially between 3 and 6. Then, the solutions are, in general, purified by passing through a filter, for example with a pore size of 0.22 μm, which makes it possible to envisage sterilizing filtration. The particles are formed by mixing, under stirring, the two solutions, whose respective volumes have been established beforehand in relation to the desired value of R, representing the ratio between the positive and negative charges of the polycation and the polyanion, respectively. The dispersion thus obtained can then be centrifuged, for example for a period between 10 minutes and 90 minutes, preferentially between 30 minutes and 70 minutes. The rate of centrifugation, for example, is between 600 g and 20,000 g (g corresponding to Earth's gravity), preferentially between 4,000 g and 15,000 g, and even more preferentially between 6,000 g and 12,000 g. Lastly, the particles can then be redispersed in the desired medium, with a solids content notably between 0.1% and 5%, preferentially between 0.5% and 3%, and even more preferentially between 0.8% and 2%. The entire method can be implemented at room temperature and under atmospheric pressure.

The particles of the invention can be provided in the form of a powder obtained, for example, after a lyophilization step, or in the form of an aqueous-phase colloidal solution with, for example, a pH in the range of 2 to 9, preferentially in the range of 4 to 8, and notably with a solids content (total mass of polymers adjusted to 100 ml of colloidal solution) in the range of 0.01% to 5%, preferentially in the range of 0.1% to 2%. Such a colloidal solution may contain one or more salts, for example sodium chloride (NaCl), with a total salts concentration of, preferably, at most 400 mM. Such a colloidal solution containing salts and/or at physiological pH (7.4) is stable at room temperature for a sufficient length of time, in particular for at least 20 days, permitting its use in biological applications in particular. A longer period of stability can be obtained, notably for its storage, at a lower temperature.

In order to obtain one such colloidal dispersion, the particles can undergo one or more operations, notably to attain the desired solids content. The particles can be separated in the aqueous phase in which they are obtained, washed and redispersed in another aqueous phase, or the colloidal solution obtained can be concentrated in order to attain the desired particles content.

The particles of the invention may include a compound of interest or an active agent. As examples of an active agent, particular mention may be made of compounds of interest in therapeutics (active organic molecule), proteins, nucleic acids, hormones, vitamins, compounds of interest in cosmetics such as perfumes, for example, fragrances, etc. The aforesaid compound of interest or active ingredient will be associated with the particles by encapsulation during the synthesis of the particles (the compound of interest being added either to the polyanion solution or to the polycation solution) by adsorption at the interface of preformed particles or by diffusion inside preformed particles.

As examples of application, the particles of the invention can be used for the preparation of a pharmaceutical, cosmetic, dermatological or dietary composition.

The examples below serve to illustrate the invention without being restrictive. The reference techniques in the context of the invention, to determine the characteristics of the polymers and the particles, are also given.

Determination of Molar Masses

The average molecular mass by weight (Mw) and the polydispersity index (PDI) of the polymers are determined by steric exclusion chromatography coupled on-line with a differential refractometer (Waters 410) and with a multi-angle laser light scattering (MALLS) system (Dawn, DSP, operational wavelength 632.8 nm). The light scattering data are analyzed using the Rayleigh-Debye equation. The refractive index increments (dn/dc) are determined for each sample with an interferometer (NFT Scan Ref) at a wavelength of 632.8 nm.

Conditions for the chromatographic analyses of chitosan: TSK 3000 and 6000 columns are used in a high-performance liquid chromatography (HPLC) system with the following buffer as eluent: acetic acid (0.2 M)/ammonium acetate (0.15 M), pH 4.5, degassed beforehand. The flow rate is 0.5 ml·min⁻¹.

Conditions for the chromatographic analyses of polyanions: A column (aquagel-OH 5, Polymer Laboratories) is used and the eluent is aqueous NaNO₃ solution (0.1M, pH 7).

Since any polymer is composed of a distribution of chains of variable lengths, average molar mass by weight is defined by the following formula known to the person skilled in the art:

$${\overline{M}}_{w} = \frac{Σ {n}_{x} {M}_{x}^{2}}{Σ {n}_{x} {M}_{x}}$$

where x is the degree of polymerization, nₓ is the number of macromolecules of degree of polymerization x and Mₓ is the mass of such macromolecules.

This size is determined by various methods known to the person skilled in the art.

Determination of the Charge Densities of the Polyelectrolytes

Determination for chitosan: This rests on the determination of the degree of acetylation (DA), which represents the percentage of N-acetylglucosamine units in the macromolecular chain. The DA is determined by proton nuclear magnetic resonance (¹H NMR) measurement of the intensity of the resonance signal of the protons of the methyl groups with that of the protons of the ring (H2-H6), situated between 3 and 4 ppm. The degree of acetylation is then determined by the following relationship:

$$DA ( \% ) = \frac{1 \text{/} 3 \times {I}_{{CH}_{3}}}{1 \text{/} 6 \times {I}_{( H 2 - H 6 )}} \times 100$$

This method is known as the Hirai method (Hirai A. et al. Polym Bull. 1991, 26, 87).

Determination of the charge density of the polysulfates: This rests on a colorimetric assay of the number of sulfate functional groups by means of toluidine blue and using a UV/VIS spectrophotometer (μQuant, BioTek Instruments). A standard range of dextran sulfate is prepared by making a range of concentrations of sulfate functional groups of 7×10⁻⁵ to 1.4×10⁻⁴ M in sodium acetate buffer (10 mM, pH 4). Toluidine blue is added to each solution at a concentration of 10⁻⁴ M. Toluidine blue complexes with the polymer which precipitates, the titration volume of the assay corresponding to the increase in absorbance at 645 nm due to excess toluidine blue.

Determination of the Average Diameter of the Particles

The average diameter of the particles is determined by quasi-elastic light scattering using, for example, the Zetasizer HS 3000 apparatus (Malvern) and the associated expert system. The measurements provide the average hydrodynamic diameter (Dₕ) obtained by the quasi-elastic light scattering method and data processing using the cumulants method and the polydispersity index (PDI) corresponds to the formula μ2/[Γ]² where μ2 is the second cumulant of the correlation function resulting from the analysis of the data by the cumulants method and [Γ]² is the average rate of decline. A polydispersity index of less than or equal to 0.05 is characteristic of a population of similar size (monodisperse) whereas an index between 0.05 and 0.15 is representative of a wider range of sizes (polydisperse) (Coombes, A. G. A.; Scholes, R D.; Davies, M. C.; Ilium, L.; Davis S. S. Biomaterials 1994, 15, 673)

Protocol for Controlling Colloidal Stability

Once recovered in the desired medium, the particles are analyzed by quasi-elastic light scattering. The average diameter (D₀) of the particles is determined a few minutes after their redispersion (the medium will be either 150 mM aqueous sodium chloride solution or PBS buffer, Invitrogen™/GIBCO® PBS, pH 7.4, 1×, lot 712299). Next, the dispersion is stored at room temperature with no stirring. The diameter of the complexes is checked regularly. The particles are considered stable when the variation in the average diameter in relation to D₀ (average diameter of the particles in the storage medium after elaboration) is less than or equal to 40%, preferentially less than 30%, and even more preferentially less than 20%. The colloidal stability is studied at room temperature, unless stated otherwise.

a) Protocol for the Hydrolysis of Chitosan, Making it Possible to Control the Molar Mass of the Chitosan

Hydrolysis of the chitosan is carried out by nitrous deamination. The chitosan is dissolved in 0.2 M acetic acid/0.15 M ammonium acetate buffer at a concentration of 0.5% by weight (w/v). After total dissolution of the chitosan, a precise volume of sodium nitrite solution at an initial concentration of 10 g/l is added in order to obtain a nitrite/glucosamine unit molar ratio of 0.1. The duration of the hydrolysis is determined in relation to the desired molar mass of chitosan. The reaction is stopped by precipitation of the chitosan, by adding diluted ammonia to reach a pH between 9 and 11. The polymer then undergoes a series of six washings with deionized water by washing-centrifugation cycles (20 minutes at 10,000 g at 4° C.) until a neutral pH is obtained. After the final centrifugation, the water is removed and the chitosan is freeze-dried.

b) Protocol for the Reacetylation of Chitosan Making it Possible to Control the Degree of Acetylation of the Chitosan

This method is adapted from work by Vachoud et al. (L. Vachoud, N. Zydowicz, A. Domard Carbohydrate Research 302, 169-177, 1997). The chitosan is placed in solution in a volume (V) of water at a concentration equal to 1% by weight to which 4 g/l of acetic acid is added. After dissolution of the chitosan, a volume of 1,2-propanediol (Sigma-Aldrich) corresponding to 80% of V is added gradually under stirring. Stirring is maintained for 30 minutes and then the mixture is degassed for 1 hour at room temperature. A mixture corresponding to 20% of V of 1,2-propanediol and of X g of acetic anhydride, in accordance with the equation (1) below, is then added to the solution. The reaction proceeds for 12 hours. Finally, the reacetylated polymer is recovered after having undergone the same steps of precipitation, of washing with deionized water (15 washings) and of lyophilization as previously in paragraph a).

$$X = \frac{{m}_{chitosan} \times ( 1 - {\%}_{water} ) \times ( {DA}_{1} - {DA}_{0} ) \times {M}_{acetic anhydride}}{{M}_{non \text{-} acetylated} \times ( 1 - {DA}_{0} ) + {M}_{acetylated} \times {DA}_{0}} \qquad ( 1 )$$

with m_{chitosan}, the mass of chitosan introduced; %_{water}, the quantity of water contained in the chitosan (determined by TGA); M_{acetic anhydride}, the molar mass of acetic anhydride; DA₁, the final DA; DA₀, the initial DA; M_{non-acetylated}, the molar mass of the non-acetylated moiety and m_{acetylated}, the molar mass of the acetylated moiety.

### Example of Reacetylation

To prepare 5 g of chitosan with a degree of acetylation of 40%, 5 g of chitosan (DA=6%; Mw=470,000 g/mol) is solubilized in 500 ml of acetic acid solution. Once the polymer is dissolved, 400 ml of 1,2-propanediol is gradually added to the mixture. After 30 minutes of stirring, the system is degassed with air for one hour. Next, 20 ml of 1,2-propanediol containing 0.95 g of acetic anhydride is added to the reaction medium.

### EXAMPLE 1

### Chitosan of DA 44%, Mw 70 kg/mol+Dextran Sulfate of 500 kg/mol

106 mg of a chitosan (France chitin, lot 113) whose degree of acetylation (DA) and average molar mass by weight (Mw) are equal to 44% and 70 kg/mol, respectively, is placed in solution under magnetic stirring in 93 g of water (Water for irrigation, Versol) containing 105 μl of glacial acetic acid (Sigma-Aldrich) and 273 mg of sodium chloride (NaCl, Sigma-Aldrich). 32 mg of dextran sulfate sodium salt (Dextran sulfate sodium salt from Leuconostoc spp., Sigma-Aldrich) whose minimal average molar mass by weight is equal to 500 kg/mol is solubilized in 30.35 g of water containing 87 mg of NaCl. The solutions are maintained under stirring for 16 hours. In order to adjust the pH of the solutions to 4, 100 μl of 0.1 M sodium hydroxide solution (NaOH, Sigma-Aldrich) and then 5 μl of 0.01 M hydrochloric acid (Sigma-Aldrich) are added to the chitosan solution and the dextran sulfate (DS) solution. The pH of the solutions is monitored using a Hanna HI 207 pH meter. The pH values for the chitosan and DS solutions are 4 and 4.4, respectively. These solutions are purified using a syringe and a 0.22 μm filter (Millipore, MILLEX®GP, 0.22 μm). Next, 15 g of the chitosan solution and 3.9 g of the DS solution are sampled. The charge ratio between the chitosan and the dextran sulfate is equal to 2 (corresponding to total ionization of the two polyelectrolytes). The DS solution is added to the chitosan solution under strong magnetic stirring. The dispersion thus obtained is centrifuged at 10,000 g for 60 minutes (BECKMAN J2-21 centrifuge, JA-20 rotor). Once the supernatant is discarded, the nanoparticles are redispersed in 300 μl of PBS (Invitrogen™/GIBCO® PBS, pH 7.4, 1×, lot 712299). The average diameter of the particles (D₀) is determined by quasi-elastic light scattering (Nano ZS®, Malvern Instruments) and is equal to 340 nm. The polydispersity index (PDI) is equal to 0.17.

The colloidal stability of the nanoparticles elaborated in example 1 is evaluated by following the evolution of the diameter of the particles over time. The particles are stored at room temperature (22° C.) in PBS in a 2 ml test tube (Eppendorf) with no stirring and their average diameter is measured at regular intervals. The following table shows the evolution of the average diameter and the PDI of the nanoparticles over 63 days.

| Storage period | Average | |
|---|---|---|
| (days) | diameter (nm) | PDI |
| 0 (D₀) | 340 | 0.17 |
| 17 | 399 | 0.25 |
| 29 | 408 | 0.23 |
| 63 | 457 | 0.24 |

### EXAMPLE 2

### Chitosan of DA 44%, Mw 70 kg/mol+Dextran Sulfate of 5 kg/mol

The nanoparticles are elaborated according to the same protocol as that described in example 1. The composition of the chitosan solution is identical to that of example 1. 32.3 mg of dextran sulfate sodium salt (Dextran sulfate sodium salt from Leuconostoc spp., Sigma-Aldrich) whose average molar mass is equal to 5 kg/mol is solubilized in 30.2 g of water containing 87 mg of NaCl. The diameter (D₀) and the PDI of the particles obtained after redispersion in PBS are equal to 327 nm and 0.13, respectively.

The colloidal stability of the nanoparticles elaborated in example 2 is evaluated according to the method of example 1. The following table shows the evolution of the average diameter and the PDI of the nanoparticles over 63 days.

| Storage period | Average | |
|---|---|---|
| (days) | diameter (nm) | PDI |
| 0 (D₀) | 400 | 0.13 |
| 17 | 400 | 0.08 |
| 29 | 383 | 0.09 |
| 63 | 353 | 0.19 |

### EXAMPLE 3

### Chitosan of DA 48%, Mw 130 kg/mol+Dextran Sulfate of 500 kg/mol

The nanoparticles are elaborated according to the same protocol as that described in example 1. 70 mg of a chitosan whose DA and molar mass (Mw) are equal to 48% and 130 kg/mol, respectively, is placed in solution under magnetic stirring in 60.12 g of water (Water for irrigation, Versol) containing 80 μl of glacial acetic acid and 180 mg of NaCl. 55 mg of dextran sulfate sodium salt (Dextran sulfate sodium salt from Leuconostoc spp., Sigma-Aldrich) whose average minimal molar mass is equal to 500 kg/mol is solubilized in 50 g of water containing 146.5 mg of NaCl. 15 g of chitosan solution and 3.7 g of DS solution are then sampled. The charge ratio between the chitosan and the dextran sulfate is equal to 3. The nanoparticles obtained after redispersion in PBS have an average diameter and a PDI equal to 407 nm and 0.19, respectively.

The colloidal stability of the nanoparticles elaborated in example 3 is evaluated according to the method of example 1. The following table shows the evolution of the average diameter and the PDI of the nanoparticles over 91 days.

| | Average diameter | |
|---|---|---|
| Storage period (days) | (nm) | PDI |
| 0 (D₀) | 407 | 0.19 |
| 10 | 398 | 0.15 |
| 28 | 368 | 0.15 |
| 45 | 382 | 0.17 |
| 91 | 375 | 0.17 |

### EXAMPLE 4

### Chitosan of DA 48%, Mw 130 kg/mol+Dextran Sulfate of 5 kg/mol

The nanoparticles are elaborated according to the same protocol as that described in example 1. 36.5 mg of a chitosan identical to example 3 is placed in solution under magnetic stirring in 30 g of water (Water for irrigation, Versol) containing 35 μl of glacial acetic acid and 89.3 mg of NaCl. 34.5 mg of dextran sulfate sodium salt (Sigma-Aldrich) whose average molar mass is equal to 5 kg/mol is solubilized in 30 g of water containing 85.9 mg of NaCl. 15 g of chitosan solution and 2.4 g of DS solution are then sampled. The charge ratio between the chitosan and the dextran sulfate is equal to 3. The nanoparticles obtained after redispersion in PBS have an average diameter and a PDI equal to 415 nm and 0.17, respectively.

The colloidal stability of the nanoparticles elaborated in example 4 is evaluated by following the method of example 1. The following table shows the evolution of the average diameter and the PDI of the nanoparticles over 111 days.

| Storage period | Average | |
|---|---|---|
| (days) | diameter (nm) | PDI |
| 0 (D₀) | 415 | 0.17 |
| 5 | 430 | 0.19 |
| 19 | 407 | 0.16 |
| 47 | 376 | 0.13 |
| 65 | 370 | 0.13 |
| 111 | 375 | 0.17 |

### EXAMPLE 5

### Chitosan of DA 48%, Mw 130 kg/mol+Heparin

The nanoparticles are elaborated according to the protocol described in example 1. 36.2 mg of a chitosan whose DA and molar mass are equal to 48 and 130 kg/mol, respectively, is placed in solution under magnetic stirring in 32.2 g of water (Water for irrigation, Versol®) containing 30 μl of glacial acetic acid and 95 mg of NaCl. 32.2 mg of sodium heparin (Heparin sodium salt from porcine intestinal mucosa, product number H4784, Sigma-Aldrich) of 9 to 12 kg/mol is solubilized in 32.02 g of water containing 89 mg of NaCl. 15 g of chitosan solution and 4.5 g of heparin solution are then sampled. The charge ratio between the chitosan and the polyanion is equal to 2. The nanoparticles obtained after redispersion in a solution (Versol® water, lot 3007088) containing 150 mM NaCl have an average diameter and a PDI equal to 440 nm and 0.17, respectively.

The colloidal stability of the nanoparticles produced is evaluated according to the method of example 1. The following table shows the evolution of the average diameter and the PDI of the nanoparticles over 20 days.

| Storage period | Average diameter | |
|---|---|---|
| (days) | (nm) | PDI |
| 0 (D₀) | 440 | 0.17 |
| 4 | 425 | 0.13 |
| 14 | 427 | 0.10 |
| 20 | 272 | 0.18 |

### COMPARATIVE EXAMPLES

The examples presented below were obtained according to the procedure of example 1. Only the properties of the chitosan (molar mass and DA) and the molar mass of the dextran sulfates were varied as stipulated in the table below. None of these formulations leads to stable colloids, and in many cases no particles are formed.

| | | | | | | | |
|---|---|---|---|---|---|---|---|
| DA | 5 | 30 | 45 | 45 | 47 | 51 | 71 |
| Chitosan | | | | | | | |
| Mw | 130 | 120 | 39 | 180 | 290 | 150 | 200 |
| Chitosan | | | | | | | |
| (kg/mol) | | | | | | | |
| Mw | 500 | 500 | 500 | 500 | 500 | 500 | 500 |
| Dextran | | 10 | 5 | 5 | | 10 | |
| sulfate | | | | | | | |
| (kg/mol) | | | | | | | |

| | | | |
|---|---|---|---|
| Stability at | No formation of particles | *NO | *NO |
| 20 days | | | |
| PBS or | | | |
| *150 mM | | | |
| NaCl | | | |

### EXAMPLE 7

### Chitosan of DA 47%, Mw 70 kg/mol+Chondroitin Sulfate

The nanoparticles are elaborated according to the same protocol as that described in example 1. 173 mg of a chitosan whose DA and molar mass are equal to 47 and 70 kg/mol, respectively, is placed in solution under magnetic stirring in 150 g of water (Water for irrigation, Versol) containing 50 μl of glacial acetic acid and 440 mg of NaCl. 35 mg of chondroitin sulfate (Chondroitin 4-sulfate sodium salt from bovine trachea, Sigma-Aldrich) is solubilized in 30 g of water containing 88 mg of NaCl. 15 g of chitosan solution and 5.5 g of chondroitin sulfate solution are then prepared. The charge ratio between the chitosan and the polyanion is equal to 2.8. The nanoparticles obtained after redispersion in 150 mM NaCl solution have an average diameter and a PDI equal to 275 nm and 0.14, respectively.

The colloidal stability of the nanoparticles elaborated in example 7 is evaluated according to the method of example 1. The following table shows the evolution of the average diameter and the PDI of the nanoparticles over 20 days.

| Storage period | Average | |
|---|---|---|
| (days) | diameter (nm) | PDI |
| 0 | 275 | 0.14 |
| 20 | 262 | 0.17 |

### EXAMPLE 8

### Chitosan of DA 42%, Mw 84 kg/mol and Heparin

The nanoparticles are elaborated according to the same protocol as that described in example 1. 94 mg of a chitosan whose DA and molar mass are equal to 42 and 84 kg/mol, respectively, is placed in solution under magnetic stirring in 82 g of water (Water for irrigation, Versol®) containing 70 μl of glacial acetic acid and 240 mg of NaCl. 32 mg of sodium heparin (Heparin sodium salt from porcine intestinal mucosa, product number H4784, Sigma-Aldrich) is solubilized in 30 g of water containing 90 mg of NaCl. 20 g of chitosan solution and 6.7 g of heparin solution are then sampled. The charge ratio between the chitosan and the polyanion is equal to 2. The nanoparticles obtained after redispersion, with a solids content equal to 0.5% in 150 mM NaCl solution, have an average diameter and a PDI equal to 288 nm and 0.16, respectively.

The colloidal stability of the nanoparticles elaborated in example 8 is evaluated according to the method of example 1. The following table shows the evolution of the average diameter and the PDI of the nanoparticles over 70 days.

| | Average | |
|---|---|---|
| Storage period | diameter | |
| (days) | (nm) | PDI |
| 0 | 288 | 0.16 |
| 8 | 245 | 0.11 |
| 12 | 270 | 0.16 |
| 35 | 222 | 0.06 |
| 42 | 222 | 0.04 |
| 53 | 226 | 0.003 |
| 70 | 219 | 0.05 |

### EXAMPLE 9

### Chitosan of DA 45%, Mw 127 kg/mol+Heparin

The nanoparticles are elaborated according to the same protocol as that described in example 1. 95 mg of a chitosan whose DA and molar mass are equal to 45 and 127 kg/mol, respectively, are placed in solution under magnetic stirring in 82 g of water (Water for irrigation, Versol®) containing 70 μl of glacial acetic acid and 240 mg of NaCl. 32 mg of sodium heparin (Heparin sodium salt from porcine intestinal mucosa, product number H4784, Sigma-Aldrich) is solubilized in 30 g of water containing 90 mg of NaCl. 20 g of chitosan solution and 6.7 g of heparin solution are then sampled. The charge ratio between the chitosan and the polyanion is equal to 2. The nanoparticles obtained after redispersion with a solids content equal to 0.5% in 150 mM NaCl solution have an average diameter and a PDI equal to 306 nm and 0.17, respectively.

The colloidal stability of the nanoparticles elaborated in example 9 is evaluated according to the method of example 1. The following table shows the evolution of the average diameter and the PDI of the nanoparticles over 78 days.

| Storage period | Average | |
|---|---|---|
| (days) | diameter (nm) | PDI |
| 0 | 266 | 0.11 |
| 8 | 229 | 0.1 |
| 12 | 246 | 0.1 |
| 35 | 210 | 0.05 |
| 42 | 215 | 0.01 |
| 53 | 218 | 0.05 |
| 70 | 209 | 0.04 |
| 78 | 199 | 0.04 |

### EXAMPLE 10

### Stability at 4° C. (Chitosan of DA 42%, Mw 84 kg/mol and Dextran Sulfate of Mw 500 kg/mol)

The nanoparticles are elaborated according to the same protocol as that described in example 1. 141 mg of a chitosan whose DA and molar mass (Mw) are equal to 42% and 84 kg/mol, respectively, is placed in solution under magnetic stirring in 122 g of water (Water for irrigation, Versol®) containing 120 μl of glacial acetic acid and 355 mg of NaCl. 40 mg of dextran sulfate sodium salt (Dextran sulfate sodium salt from Leuconostoc spp., Sigma-Aldrich) whose average minimal molar mass is equal to 500,000 g/mol is solubilized in 35 g of water containing 102 mg of NaCl. 30 g of chitosan solution and 8 g of DS solution are then sampled. The charge ratio between the chitosan and the dextran sulfate is equal to 2. The nanoparticles obtained after redispersion in PBS (GIBCO® PBS, pH 7.4, 1×) with a solids content of 2% have an average diameter and a PDI equal to 339 nm and 0.17, respectively.

The colloidal stability of the nanoparticles elaborated in example 10 is evaluated according to the method of example 1. The following table shows the evolution of the average diameter and the PDI of the nanoparticles over 143 days at room temperature.

| | Average | |
|---|---|---|
| Storage period (days) | diameter (nm) | PDI |
| 0 | 339 | 0.17 |
| 9 | 328 | 0.18 |
| 35 | 327 | 0.18 |
| 87 | 306 | 0.2 |
| 97 | 315 | 0.18 |
| 121 | 333 | 0.17 |
| 143 | 384 | 0.2 |

The following table shows the evolution of the average diameter and the PDI of the nanoparticles over 143 days at 4° C.

| Storage period | Average | |
|---|---|---|
| (days) | diameter (nm) | PDI |
| 0 | 339 | 0.17 |
| 10 | 321 | 0.14 |
| 35 | 323 | 0.17 |
| 90 | 322 | 0.19 |
| 97 | 311 | 0.18 |
| 121 | 334 | 0.16 |
| 143 | 354 | 0.17 |

### EXAMPLE 11

### Stability at 4° C. (Chitosan of DA 42%, Mw 84 kg/mol and Dextran Sulfate of Mw 5,000 g/mol)

The nanoparticles are elaborated according to the same protocol as that described in example 1. 140 mg of a chitosan whose DA and molar mass (Mw) are equal to 42% and 84 kg/mol, respectively, is placed in solution under magnetic stirring in 122 g of water (Water for irrigation, Versol®) containing 120 μl of glacial acetic acid and 354 mg of NaCl. 40 mg of dextran sulfate sodium salt (Dextran sulfate sodium salt from Leuconostoc spp., Sigma-Aldrich) whose average molar mass is equal to 5,000 g/mol is solubilized in 35 g of water containing 102 mg of NaCl. 30 g of chitosan solution and 7.7 g of DS solution are then sampled. The charge ratio between the chitosan and the dextran sulfate is equal to 2. The nanoparticles obtained after redispersion in PBS (GIBCO® PBS, pH 7.4, 1×) with a solids content of 1.6% have an average diameter and a PDI equal to 358 nm and 0.18, respectively.

The colloidal stability of the nanoparticles elaborated in example 11 is evaluated according to the method of example 1. The following table shows the evolution of the average diameter and the PDI of the nanoparticles over 143 days at room temperature.

| Storage period | Average | |
|---|---|---|
| (days) | diameter (nm) | PDI |
| 0 | 358 | 0.18 |
| 9 | 307 | 0.14 |
| 35 | 310 | 0.13 |
| 87 | 273 | 0.17 |
| 97 | 282 | 0.13 |
| 121 | 305 | 0.12 |
| 143 | 346 | 0.17 |

The following table shows the evolution of the average diameter and the PDI of the nanoparticles over 143 days at 4° C.

| Storage period | Average | |
|---|---|---|
| (days) | diameter (nm) | PDI |
| 0 | 358 | 0.18 |
| 10 | 348 | 0.17 |
| 35 | 321 | 0.15 |
| 90 | 359 | 0.17 |
| 97 | 319 | 0.15 |
| 121 | 338 | 0.17 |
| 143 | 359 | 0.19 |

### EXAMPLE 12

### Stability at 37° C. (Chitosan of DA 42%, Mw 84 kg/mol and Dextran Sulfate of Mw 500 kg/mol)

The nanoparticles are elaborated according to the same protocol as that described in example 1. 141 mg of a chitosan whose DA and molar mass (Mw) are equal to 42% and 84 kg/mol, respectively, is placed in solution under magnetic stirring in 122 g of water (Water for irrigation, Versol®) containing 120 μl of glacial acetic acid and 356 mg of NaCl. 86 mg of dextran sulfate sodium salt (Dextran sulfate sodium salt from Leuconostoc spp., Sigma-Aldrich) whose average minimal molar mass is equal to 500,000 g/mol is solubilized in 80 g of water containing 234 mg of NaCl. 30 g of chitosan solution and 8.6 g of DS solution are then sampled. The charge ratio between the chitosan and the dextran sulfate is equal to 2. The nanoparticles obtained after redispersion in 150 mM NaCl solution (Versol® water) with a solids content of 1%, have an average diameter and a PDI equal to 354 nm and 0.24, respectively.

The colloidal stability of the nanoparticles elaborated in example 12 is evaluated according to the method of example 1. The following table shows the evolution of the average diameter and the PDI of the nanoparticles over 79 days at 37° C.

| Storage period | Average | |
|---|---|---|
| (days) | diameter (nm) | PDI |
| 0 | 354 | 0.24 |
| 1 | 326 | 0.21 |
| 6 | 293 | 0.19 |
| 13 | 258 | 0.17 |
| 20 | 260 | 0.16 |
| 29 | 314 | 0.28 |
| 60 | 215 | 0.06 |
| 79 | 210 | 0.06 |

### EXAMPLE 13

### Stability at 37° C. (Chitosan of DA 42%, Mw 84 kg/mol and Dextran Sulfate of Mw 5,000 g/mol)

The nanoparticles are elaborated according to the same protocol as that described in example 1. 141 mg of a chitosan whose DA and molar mass (Mw) are equal to 42% and 84 kg/mol, respectively, are placed in solution under magnetic stirring in 122 g of water (Water for irrigation, Versol®) containing 120 μl of glacial acetic acid and 356 mg of NaCl. 86 mg of dextran sulfate sodium salt (Dextran sulfate sodium salt from Leuconostoc spp., Sigma-Aldrich) whose average molar mass is equal to 5,000 g/mol is solubilized in 80 g of water containing 237 mg of NaCl. 30 g of chitosan solution and 8.3 g of DS solution are then sampled. The charge ratio between the chitosan and the dextran sulfate is equal to 2. The nanoparticles obtained after redispersion in 150 mM NaCl solution (Versol® water) with a solids content of 1% have an average diameter and a PDI equal to 297 nm and 0.13, respectively.

The colloidal stability of the nanoparticles elaborated in example 13 is evaluated according to the method of example 1. The following table shows the evolution of the average diameter and the PDI of the nanoparticles over 79 days at 37° C.

| | Average diameter | |
|---|---|---|
| Storage period (days) | (nm) | PDI |
| 0 | 297 | 0.13 |
| 1 | 261 | 0.13 |
| 6 | 256 | 0.08 |
| 13 | 227 | 0.1 |
| 20 | 243 | 0.09 |
| 34 | 232 | 0.05 |
| 60 | 219 | 0.03 |
| 79 | 222 | 0.04 |
| 141 | 198 | 0.04 |

### EXAMPLE 14

### Chitosan of DA 48%, Mw 130,000 g/mol+Heparan Sulfate

The nanoparticles are elaborated according to the protocol described in example 1. 36.2 mg of a chitosan whose DA and molar mass are equal to 48 and 130 kg/mol, respectively, are placed in solution under magnetic stirring in 322 g of water (Water for irrigation, Versol®) containing 30 μl of glacial acetic acid and 95 mg of NaCl. 34 mg of heparan sulfate (Heparan sulfate sodium salt from bovine kidney, product number H7640, Sigma-Aldrich) is solubilized in 32.02 g of water containing 89 mg of NaCl. 15 g of chitosan solution and 4.5 g of a heparan sulfate solution are then sampled. The charge ratio between the chitosan and the polyanion is equal to 2. The nanoparticles obtained after redispersion in a solution (Versol® water, lot 3007088) containing 150 mM NaCl have an average diameter and a PDI equal to 430 nm and 0.14, respectively.

The colloidal stability of the nanoparticles produced is evaluated according to the method of example 1. The following table shows the evolution of the average diameter and the PDI of the nanoparticles over 20 days.

| Storage period | Average diameter | |
|---|---|---|
| (days) | (nm) | PDI |
| 0 (D₀) | 430 | 0.14 |
| 4 | 414 | 0.13 |
| 14 | 427 | 0.10 |
| 20 | 272 | 0.18 |

1- Positively charged particles consisting of a chitosan polyelectrolyte complex and of an anionic polymer, characterized in that the chitosan has a degree of acetylation (DA) in the range of 35 to 49% and a mean molar mass by weight (Mw) in the range of 55 to 150 kg/mol, and in that the anionic polymer is selected from hyaluronic acid, dextran sulfate, cellulose sulfate, chondroitin sulfate, heparan sulfate, dermatan sulfate, keratan sulfate, alginates, pectins, carboxymethyl dextran, carboxymethyl amylose, carboxymethyl cellulose, carboxymethyl beta-cyclodextrin, heparin, polystyrene sulfonate, linear or branched water-soluble synthetic homo- or co-polymers containing at least one anionic monomer having either a carboxyl functional group or a sulfonic acid functional group and optionally one or more nonionic monomers.

2- The particles according to claim 1, characterized in that the chitosan has a degree of acetylation (DA) in the range of 45 to 48% and a mean molar mass by weight (Mw) in the range of 70 to 130 kg/mol.

3- The particles according to claim 1, characterized in that the anionic polymer is dextran sulfate.

4- The particles according to claim 1, characterized in that the ratio between the number of charges of the chitosan and the number of charges of the anionic polymer (n⁺/n⁻) is in the range of 1.05 to 5, preferably in the range of 1.5 to 3.

5- The particles according to claim 1, characterized in that the average diameter of the particles is in the range of 50 nm to 50 μm, preferably in the range of 150 nm to 5 μm.

6- The particles according to claim 1, characterized in that they are obtained by the addition of an aqueous solution of the chitosan or of the anionic polymer to an aqueous solution of the other polymer (polyanion or chitosan), said solutions being at a pH in the range of 2 to 9, preferentially in the range of 3 to 8.

7- The particles according to claim 6, characterized in that at least one of said solutions contains a monovalent salt at most at a concentration of 400 mM, preferentially at most 150 mM, for example in the form of NaCl.

8- The particles according to claim 1, characterized in that they include an active agent.

9- The particles according to claim 1, characterized in that they are provided in the form of an aqueous-phase colloidal dispersion with a pH in the range of 2 to 9, preferentially in the range of 4 to 8, with a solids content (total mass of polymers adjusted to 100 ml of colloidal solution) in the range of 0.01% to 5%, preferentially in the range of 0.1% to 2%.

10- The particles according to claim 9, characterized in that the colloidal solution contains one or more salts, for example NaCl, the total salt concentration being at most equal to 400 mM.

11- A method for preparing particles according to claim 1, which includes the following steps:
a) have available an aqueous solution of chitosan,
b) have available an aqueous solution of anionic polymer selected from hyaluronic acid, dextran sulfate, cellulose sulfate, chondroitin sulfate, heparan sulfate, dermatan sulfate, keratan sulfate, alginates, pectins, carboxymethyl dextran, carboxymethyl amylose, carboxymethyl cellulose, carboxymethyl beta-cyclodextrin, heparin, polystyrene sulfonate, linear or branched water-soluble synthetic homo- or co-polymers containing at least one anionic monomer having either a carboxyl functional group or a sulfonic acid functional group and optionally one or more nonionic monomers,
c) add one of these solutions to the other solution so as to obtain a colloidal solution of positively charged particles consisting of a chitosan polyelectrolyte complex and of a polyanion.

12- The method for preparing particles according to claim 11, characterized in that the chitosan has a degree of acetylation (DA) in the range of 35 to 49% and a mean molar mass by weight (Mw) in the range of 55 to 150 kg/mol.

13- The method for preparing particles according to claim 11, characterized in that the anionic polymer is dextran sulfate.

14- The method for preparing particles according to claim 11, characterized in that the ratio between the number of charges of the chitosan and the number of charges of the anionic polymer (n⁺/n⁻) is in the range of 1.05 to 5, preferably in the range of 1.5 to 3.

15- The method for preparing particles according to claim 11, characterized in that both the chitosan solution and the anionic polymer solution have a pH in the range of 2 to 9, preferentially in the range of 3 to 8.

16- The method for preparing particles according to claim 11, characterized in that the chitosan solution or the anionic polymer solution contains a monovalent salt at most at a concentration of 400 mM, preferentially at most 150 mM.

17- The method for preparing particles according to claim 11, characterized in that the aqueous solution of chitosan is added to the aqueous solution of anionic polysaccharide.

18- The method for preparing particles according to claim 11, characterized in that the particles are separated from the aqueous phase in which they are obtained, washed and redispersed in another aqueous phase.
